(19) United States
(12) Patent Application Publication
(10) Pub. No.: US 2023/0295682 A1
(43) Pub. Date: Sep. 21, 2023

(54) METHOD AND SYSTEM FOR RAPID DETECTION OF LOW LEVEL BACTERIA IN A GROWTH MEDIUM

(71) Applicants: William Alexander ANDERSON, Waterloo (CA); Shazia TANVIR, Waterloo (CA)

(72) Inventors: William Alexander ANDERSON, Waterloo (CA); Shazia TANVIR, Waterloo (CA)

(21) Appl. No.: 18/010,919

(22) PCT Filed: Jun. 18, 2021
(86) PCT No.: PCT/CA2021/050834

§ 371 Date: Dec. 16, 2022

Related U.S. Application Data
(60) Provisional application No. 63/102,510, filed on Jun. 18, 2020.

(51) Int. Cl. C12Q 1/10; C12Q 1/22

(52) U.S. Cl. CPC C12Q 1/10; C12Q 1/22

(57) ABSTRACT

The disclosure is directed at a system and method for rapid detection of low level bacteria in a growth medium. The method is directed at a two-stage process whereby a growth medium is incubated along with a first test formulation component for a predetermined period of time. After the incubation period, a second test formulation component is added to the growth medium to determine if there is a bacteria in the growth medium.

### CROSS-REFERENCE TO OTHER APPLICATIONS

The disclosure claims the benefit of priority from U.S. Provisional Application No. 63/102,510 filed June 18th 2020, which is hereby incorporated by reference.

### FIELD

The disclosure is generally directed at bacteria detection, and more specifically, at a method and system for rapid detection of low level bacteria in a growth medium.

### BACKGROUND

Current standard detection methods for Ecoli contamination in water require the shipment of samples to a laboratory, where a plating culture method is then used to test the water sample or samples. This method typically uses membrane filtration of the water sample, followed by incubation of the filter on a selective agar plate at a specified temperature. This membrane filter method typically requires one to two days for a result or determination to be produced, not including sample shipment time (which can also affect the results). This leaves a period of time where the water quality is uncertain or unknown, exposing water users to risk.

Other methods of measuring Ecoli are known, such as genomic methods (PCR), however, this process is laboratory based and requires sophisticated instrumentation. Immunological methods are also used based on antibody interactions with the Ecoli, however, this methodology requires antibodies that are expensive and often not very stable in storage. Also, there is a need for expensive instrumentation such as fluorescence spectrometers. Culture methods with selective substrate testing are also used but require at least 12 to 24 hours for a visible response. Also, the detection limit (level where bacteria is detected) is quite high, and usually requires hundreds or thousands of Ecoli cells per mL (cfu/mL) where bacteria testing for water may require a detection limit of 0.1 cfu/mL or lower. Finally, another current E-coli test method is the use of biosensor methods which may be seen as electrochemical or optical methods, however, these tests generally have poor sensitivity to lower levels of Ecoli contamination such as required for water testing.

There is strong demand for a rapid test method that can be completed “in the field” in a few hours for screening purposes so that rapid intervention may be initiated if contamination exists. There is also a strong desire for a screening test method that can be performed by non-technical staff and other users with little or no equipment required, making it more accessible to a wider variety of users and situations.

Therefore, there is provided a novel system and method for rapid detection of low level bacteria in a growth medium.

### SUMMARY

The disclosure is directed at a method and system for rapid detection of bacteria in a growth medium. In one embodiment, the disclosure is directed at an ultra-sensitive enzyme-based E.coli test method that can detect low levels of bacteria within a few hours. In another embodiment, a presence of bacteria on non-liquid items may be detected by placing the item in a solid or liquid growth medium, such as an agar gel or water, and then testing the growth medium using the method and system of the disclosure.

In one embodiment, the disclosure is directed at a two-stage process, whereby a water sample is incubated in the presence of a first test component, which may be seen as a selective chromogenic substrate, for a predetermined period of time, such as a few hours, and then subject to the addition of a second test component, which may be seen as a dye reagent, to the sample.

In another aspect of the disclosure, there is provided a method of detection of low level bacteria in a growth medium sample including adding a selective chromogenic substrate to the growth medium sample; incubating the growth medium sample for a predetermined period of time; and adding a dye reagent to the growth medium sample; wherein if a color change in the growth medium sample is observed, a presence of bacteria is confirmed.

In another aspect, the selective chromogenic substrate is 5-bromo-4-chloro-3-indoxyl-beta-D-glucuronide cyclohexylammonium salt (x-gluc); 1-5-Bromo- 4-Chloro-3-Indolyl Phosphate (BCIP) or 2-5-bromo-4-chloro-3-indolyl-β-D-galactopyranoside (x-gal). In a further aspect, the dye reagent is nitroblue tetrazolium (NBT); tetrazolium-formazan substrates or resazurin substrates. In yet another aspect, before adding a selecting chromogenic substrate: placing an item of interest into the growth medium sample. In another aspect, adding a selective chromogenic substrate to the growth medium sample includes adding the selective chromogenic substrate to a liquid sample.

In yet a further aspect, adding the selective chromogenic substrate to the liquid sample includes adding the selective chromogenic substrate to a water sample. In another aspect, after adding a selective chromogenic substrate to the growth medium sample: adding nutrients to the growth medium sample to enhance bacteria growth. In a further aspect, the method includes correlating an observed color change with a level of bacteria. In yet another aspect, correlating an observed color change includes analyzing the liquid sample with a spectrophotometer or colorimeter. In a further aspect, adding a selective chromogenic substrate to the growth medium sample includes adding the selective chromogenic substrate to an agar gel sample.

In another aspect of the disclosure, there is provided a system for rapid detection of low level bacteria in a growth medium including a first test formulation component that is incubated for a predetermined period of time within the growth medium for enabling bacterial enzyme production; and a second test formulation component for adding to the incubated growth medium and first test formulation component.

In yet another aspect, the system includes a set of nutrients for enhancing bacterial enzyme production during the incubation period. In a further aspect, the growth medium is in a liquid form. In an aspect, the growth medium is in a solid form.

### DESCRIPTION OF THE DRAWINGS

Embodiments of the present disclosure will now be described, by way of example only, with reference to the attached Figures.

FIG. 1 is a flowchart outlining a method for rapid detection of low level bacteria in water;

FIG. 2 is a schematic diagram of a specific embodiment of the method of FIG. 1;

FIG. 3 is a flowchart outlining another embodiment for rapid detection of low level bacteria;

FIG. 4 are photographs showing an assay without and with NBT;

FIG. 5 is a graph showing comparison of coloration sensitivity before and after the addition of NBT for a water sample that was incubated for 6 hours at 37° C.;

FIG. 6 is a graph showing comparison of coloration sensitivity before and after the addition of NBT for a water sample that was incubated for 7 hours at 37° C.;

FIG. 7 is a graph showing comparison of coloration sensitivity before and after the addition of NBT for a water sample that was incubated for 8 hours at 37° C.;

FIG. 8 is a graph showing comparison of slope values obtained at different concentration values of E-coli incubated for 6, 7 or 8 hours at 37° C.;

FIG. 9 is a graph showing a comparison of test samples after the 6, 7 and 8 hours incubation period in a media incorporated with x-gluc at 37 C;

FIG. 10 is a graph showing the incubation of different concentrations of E-coli in the media with x-gluc for 6 hours at 37° C.;

FIG. 11 is a graph showing the incubation of different concentrations of E-coli in the media with x-gluc for 7 hours at 37° C.; and

FIG. 12 is a graph showing the incubation of different concentrations of E-coli in the media with x-gluc for 8 hours at 37° C.

### DESCRIPTION

The disclosure is directed at a method and system for rapid detection of low level bacteria in water. In some embodiments, the disclosure may be seen as a rapid test to provide an indication to the tester whether or not there are bacteria in the water being tested. One advantage of the current disclosure is that an indication with respect to the presence of bacteria in the sample may be obtained in a shorter period of time compared with current systems. Another advantage of the current disclosure is that the system and method may detect low levels of bacteria in the sample or low levels of a specific sub-set of bacteria in the sample.

This may be beneficial for the testing of water where a full study of water quality is not required or if the water being tested is in an area where sending the water sample to a laboratory may be time consuming. In other applications, the disclosure may be used to test the water in a swimming pool or beach area so that decisions with respect to the water quality may be quickly determined without the need to wait for laboratory testing.

In other embodiments of the disclosure, the disclosure may be seen as a two-stage process for low level bacteria detection in a liquid. In a specific embodiment, the method and system is directed at the detection of low level E-coli in water.

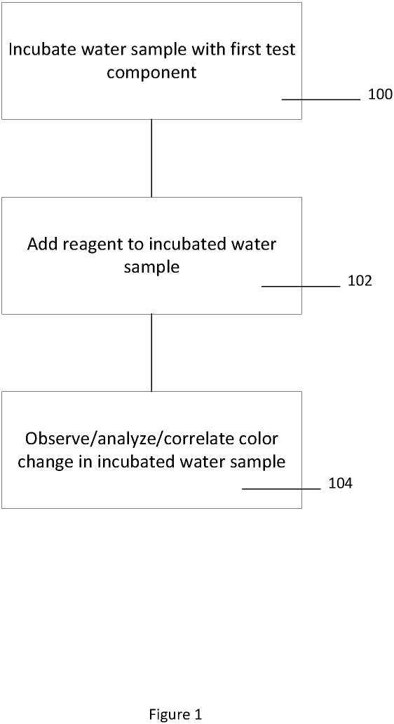

Turning to FIG. 1, a method of rapid detection of low level bacteria in a liquid growth medium, such as water, sample is shown. Initially, the liquid sample, such as a water sample, that is being tested is obtained or received. It will be understood that for bacteria, such as E-coli, to grow, the liquid may be seen as a growth medium for the bacteria.

In order to perform the method of rapid detection of low level bacteria in water, the water sample is incubated (100). In one embodiment, incubation of the water sample may be performed by adding a first test formulation component (that has been previously placed in a specific, or predetermined, media) to the water sample and then letting the water sample incubate for a predetermined period such as, but not limited to 6 to 8 hours. In other embodiments, the first test formulation component may include the specific media, and together, may form the first test formulation component. As understood, bacteria require a growth medium such as the combination of water, the first test formulation component and, possibly, certain nutrients and/or inducers to promote certain enzyme production.

In one embodiment, the first test formulation component enhances or promotes bacteria growth within the water. In another embodiment, the first test formulation component may be cleaved by an enzyme produced by the bacteria during the incubation period. Generally, the first test formulation component may be an indoxyl substrate. In a specific embodiment, the first test formulation component may be 5-bromo-4-chloro-3-indoxyl-beta-D-glucuronide cyclohexylammonium salt (x-gluc). In other embodiments, the first test formulation component may be 1-5-Bromo- 4-Chloro-3-Indolyl Phosphate (BCIP) or 2-5-bromo-4-chloro-3-indolyl-β-D-galactopyranoside (x-gal).

After the incubation period, the second test formulation component is added to the incubated water sample (102). In one example, the second test formulation component may be seen as an oxidant, or dye reagent such as, but not limited to, nitroblue tetrazolium (NBT). Other second test formulation components may include tetrazolium-formazan substrates or resazurin substrates. In the presence of bacteria, even a low level of bacteria, interacting with the dye reagent, the incubated water sample changes color whereby the color change may be observed, analyzed or correlated to determine a presence of bacteria in the water sample (104). Based on the intensity of the color change, the method of the disclosure may determine, even a low, level of bacterial in the water sample.

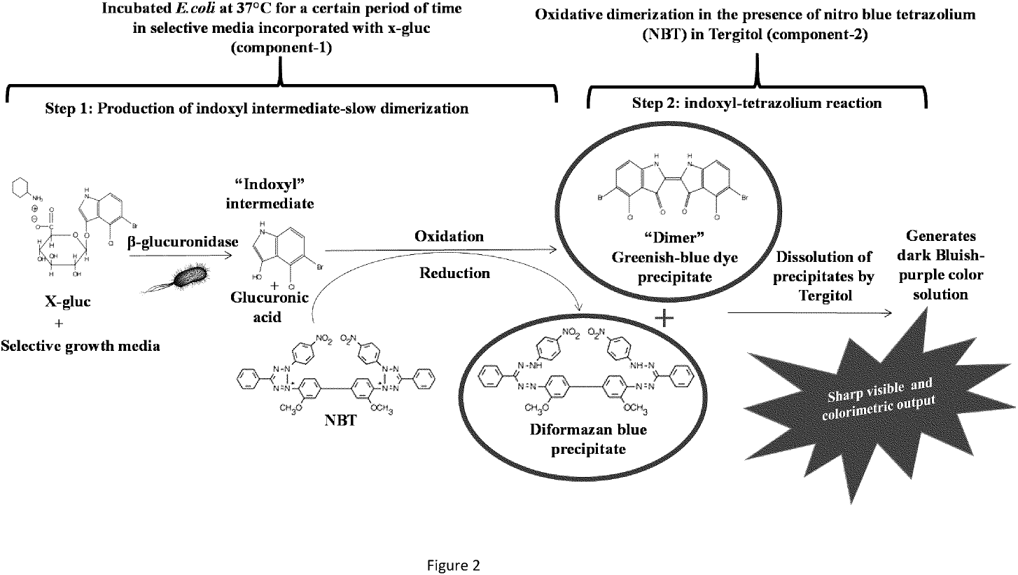

Turning to FIG. 2, a schematic diagram of a specific embodiment of FIG. 1 is shown. In FIG. 2, the bacteria is E-coli, the first test formulation component is x-gluc, and the second test component is NBT. Initially, the water sample is incubated at 37° C. with a selective media that is incorporated with the x-gluc. During the incubation period, interaction between the x-gluc and the E-coli produces an indoxyl intermediate. In one embodiment, the first test formulation component cleaves to or is cleaved by an enzyme, β-glucuronidase, which is produced by the E-coli in the water sample. In one embodiment, cleaving of the x-gluc to the enzyme produces a dimeric product. This may be seen as a first stage of the method of the disclosure.

The first test formulation component may also cause or induce the bacteria (such as E-coli ) to develop or produce an enzyme such as, β-glucuronidase. Alternatively, the inducement of increased enzyme production may be assisted via the addition of an inducer to the sample. The selection of the inducer may be dependent on the bacteria that is being detected.

After the incubation period, NBT is placed into the water sample where it may oxidize, or react, with the indoxyl intermediate. This may be seen as a second stage of the method of the disclosure. In other words, the second stage may be seen as oxidative dimerization in the presence of the NBT. The reaction between the NBT and the indoxyl intermediate may result in either a “dimer” which is a greenish-blue dye precipitate and/or a diformazan blue precipitate which produces the color change in the water sample when NBT is introduced to, even a low level of, bacteria.

The higher the amount of enzyme (or indoxyl intermediate) that is produced during the incubation period, the higher the color change intensity. In some embodiments, this color change, or the color change intensity may be correlated with a level of bacteria in the water.

In other words, during the incubation period, in the presence of E.coli, the x-gluc is converted to a different form. This different form, such as the indoxyl intermediate, chemically reacts with the second test formulation component, or NBT, to generate an intense blue colour (or a color change) within the water sample. The addition of the NBT intensifies the color of the incubated water sample through the dark-blue indigo dye formed by the oxidation of the indoxyl intermediate and the blue-purple precipitate formed by the reduction of NBT in the water sample or Tergitol.

Scientifically, bacterial growth will lead to anaerobic conditions which cause the slowdown of the oxidative dimerization process of indoxyl intermediates. The addition of the second test formulation component (such as NBT) to the medium (mixture of E-coli, water and the first test formulation component) or water sample increases the overall oxidation of indoxyl dimerization into greenish blue precipitates and it also produces a greater enhancement of color through the formation of diformazan by reduction of NBT.

In a specific experiment using the method shown schematically in FIG. 2, an observed color change was possible within 8 hours of water sample retrieval and bacteria levels as low as 10 cfu/ml were detected without the need to filtrate or concentrate the water sample (unlike current methodologies). Therefore, in this specific embodiment, a presumptive identification of E.coli within a water sample may be determined within approximately 8 h rather than 24 hours as with current laboratory methods and routines.

In a simplest form of the disclosure, the detection of any color change in the water can be used in a yes/no format with respect to a presence of, even a low level of, bacteria in the water sample. In some embodiments, the type of bacteria that is detected may depend on the growth media and the type of enzyme substrate. For example, a combination of growth media and X-gal may be used to detect coliforms while a combination of growth media and X-gluc may be used to detect E-coli .

In one embodiment, the disclosure is directed at the testing of certain bacteria that have a known inducible enzyme that will react with or cleave to the first test formulation component. The test sample, or growth medium sample, may have different types of bacteria and based on characteristics of a combination of the first test component, the second test component and the growth medium, testing may be for one or more of the different types of bacteria. The presence of a color change indicates that there is some level of bacteria or some level of the targeted bacteria in the water sample. The intensity of color change may provide an indication with respect to an approximate level of bacteria. The color change may also be scanned or read by a device such as a spectrophotometer or colorimeter to screen for Ecoli, or bacterial contamination in the water sample. In another embodiment, measurement of the color change by the spectrophotometer or colorimeter may be correlated to a bacterial level. In other potential embodiments, the color change development can be used to roughly quantify the level of bacterial contamination.

In other embodiments, the disclosure may also be used to detect other bacteria in water using other selective chromogenic substrates and dye reagents.

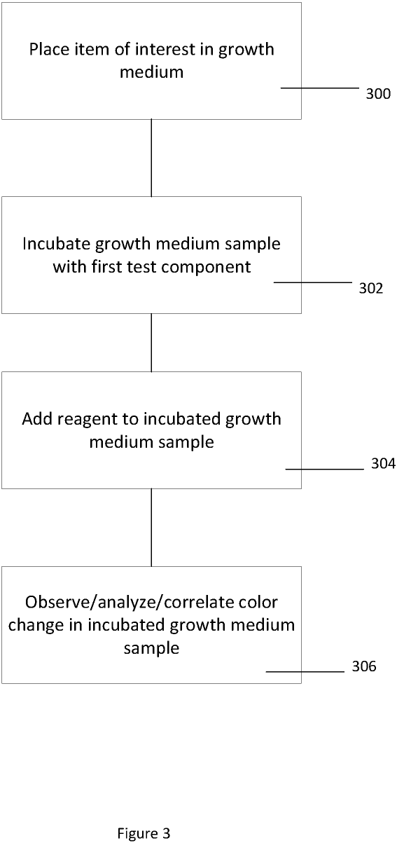

Turning to FIG. 3, another method of rapid detection of low level bacteria is shown. In the embodiment above, the embodiment was directed at the testing of water, or a water sample, to determine if there is, even a low level of, bacteria in the sample. In this embodiment, materials or surfaces may be tested to determine if there is a low level of bacteria on the material or surface. As discussed above, bacteria, such as E-coli, requires a growth medium, such as water, to be produced. Alternatively, the growth medium may be a solid, such as, but not limited to, an agar gel.

For a liquid sample, the liquid may be seen, or used, as a growth medium for the bacteria. In another embodiment, such as for the testing of low level bacteria on a surface of interest, the item of interest may be either placed in a growth medium sample, such as a container with water or a container or petri dish, and the like, with agar gel and the like. In another embodiment, a surface of interest may be swabbed, and the swab placed in a liquid or solid growth medium. The interaction of the bacteria with the growth medium promotes bacterial or enzyme growth. In yet another embodiment, soil may be placed in a liquid or solid growth medium to promote bacteria growth.

Initially, the material, or surface of interest, is placed into a container containing the growth medium, for example water or an agar gel (300). By adding the material of interest into the container including the growth medium, this may promote the growth of bacteria (assuming it was present on the surface of interest). Other nutrients may be added to the container to further promote the growth of the bacteria, although this is not necessary in each embodiment. The container of growth medium (along with the material of interest) may then be incubated (302) along with the first test formulation component for a predetermined period. After the incubation period, the second test formulation component is added to the growth medium sample (304). As discussed above, the second test formulation component may be an oxidant, or dye reagent such as, but not limited to, nitroblue tetrazolium (NBT). The growth medium sample can then be observed/analysed to determine if there is a visual color change (306). A color change within the growth medium sample provides an indication that there is a presence of bacteria on the material of interest. If a color change is visible, the color change may also be correlated to determine an approximate level of bacteria.

In some embodiments, the system and method of the disclosure may detect levels of 0.1 cfu/mL or lower in time periods of less than 8 hours. The two-stage process of the disclosure provides a detection system that both lowers the detection limit and speeds up the response or time to perform the detection.

In another specific experiment, E.coli was grown in sterilized trypticase soy broth at 37° C. for 18 hours. The cultures were then centrifuged (600 rpm), washed three times with 10 mM of sterile PBS (pH 7.4) and serially diluted. Inoculum levels were determined by using by plating onto plate count agar. Different E.coli concentrations were then prepared at 1, 10, 100 and 1000 cfµ/ml.

For the test formation used in the experiment, the specific media was produced using 11 g/L sodium phosphate Mono basic; 13 g/L sodium phosphate di basic; 10 g/L Sodium Pyruvate, 10 g/L sorbitol; 15 g/L tryptophan; 5 g/L peptone; 5 g/L yeast extract and 5 g/L sodium chloride. Other specific media will be understood by those skilled in the art. After autoclaving the media, 1000 mg/L of X-gluc was incorporated and dissolved into the specific media (“first test component”). The second test formulation component was prepared by dissolving 4 ml of Tergitol in 2000 mg/L of NBT in 1 L of deionized water (“second test component)”.

In testing the different E-coli concentrations, 200 µl of the first test component was used per ml of the E.coli dilution and 200 µl of the second test component was added per ml of E.coli solution.

During the experiment, the 200 µl of the first test component was independently mixed with the E.coli dilutions and incubated at 37° C. for the periods of 6, 7 and 8 hours.

At the end of the specific incubation time period, 200 µl of the second test component was added. After 10 minutes of interaction between the second test formulation component and the E-coli dilutions, the color change (which was present in each case) was visually observed and analyzed with a spectrophotometer. The whole experiment was also conducted using a 96 well plate to optimize the assay conditions. Negative controls were set up in each experiment using sterile phosphate-buffered saline (PBS). The analysis by the spectrophotometer may assist in the correlation of color change with respect to level of bacteria present in the test sample.

With respect to the spectrophotometer measurements, the multi-wavelength transmission spectra of E.coli tests were recorded by a UV/visible spectrophotometer in the region from 400 to 800 nm. The spectrophotometer was used to record the spectra representing the averages of three (3) replicate measurements with 1 nm sampling intervals. To eliminate, or reduce, the effect of inhomogeneity in the sample being tested, sterilized deionized water was used as a reference solution. All measurements were made using 1 cm path-length disposable cuvettes at room temperature.

The incorporation of a chromogenic substrate into selective media may facilitate identification of bacteria, improve the accuracy of the detection and/or reduce the need for isolation of pure cultures for confirmation of the presence of bacteria. For example, to accelerate E.coli detection, a first test formulation component containing the β-glucuronidase substrate x-gluc and supplements that promote the growth of E.coli and suppress the growth of unwanted bacteria were formulated.

While x-gluc reaction products and NBT have been mixed or used together in a single stage, or single step, for cell staining and histological studies using microscopy in biology, separating the two reactions sequentially provides a previously unforeseen result whereby separately adding x-gluc as part of the first test formulation component and NBT as the second test formulation component enables a novel rapid detection method for the presence of a low level of bacteria, such as Ecoli. The separation of the mixing of the two different test formulation components is necessary as NBT inhibits E.coli growth and prevents, or reduces, the likelihood of any detection from occurring if it was added at the same time with the x-gluc.

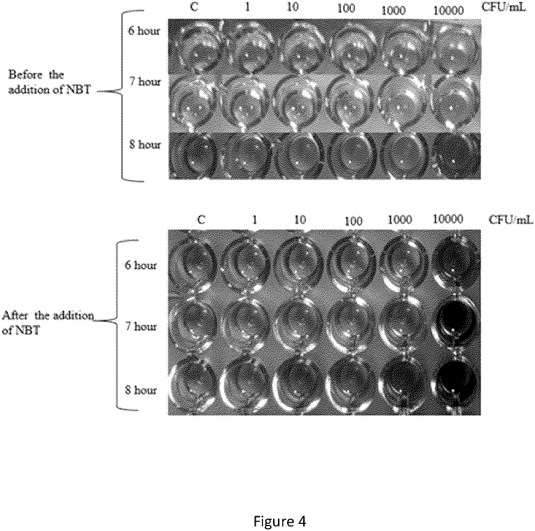

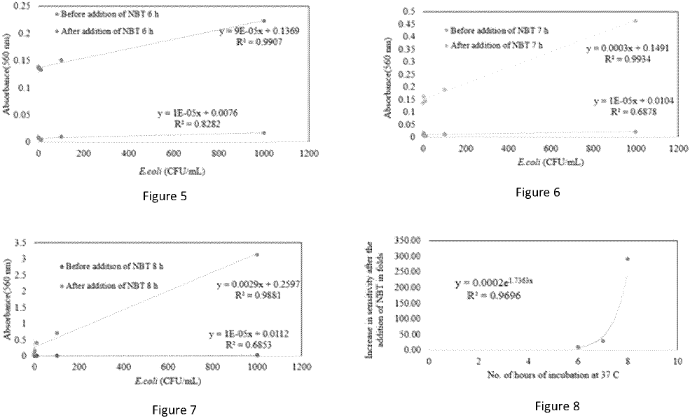

In further experiments, the method of the disclosure was tested with and without the addition NBT with results shown in the photographs of FIG. 4 which show the test assay before the addition of NBT (top photo) and the test assay 10 minutes after the addition of NBT (bottom photo). The color change was visible at lower concentration (cfu/mL) levels with the NBT versus the samples without the NBT. A color change was clearly noticed, especially with the 10000 cfu/ml solutions where an intense blue was clearly observed. Results are shown in FIGS. 5 to 8 where FIG. 5 is a comparison of coloration sensitivity before and after the addition of NBT for a water sample that was incubated for 6 hours at 37° C.; FIG. 6 is a comparison of coloration sensitivity before and after the addition of NBT for a water sample that was incubated for 7 hours at 37° C.; FIG. 7 is a comparison of coloration sensitivity before and after the addition of NBT for a water sample that was incubated for 8 hours at 37° C.; and FIG. 8 is a comparison of slope values obtained at different concentration values of E-coli incubated for 6, 7 or 8 hours at 37° C. From the graphs, it can be seen that there was an enhanced coloration sensitivity up to 290 times for the samples with NBT relative to the test performed using only x-gluc.

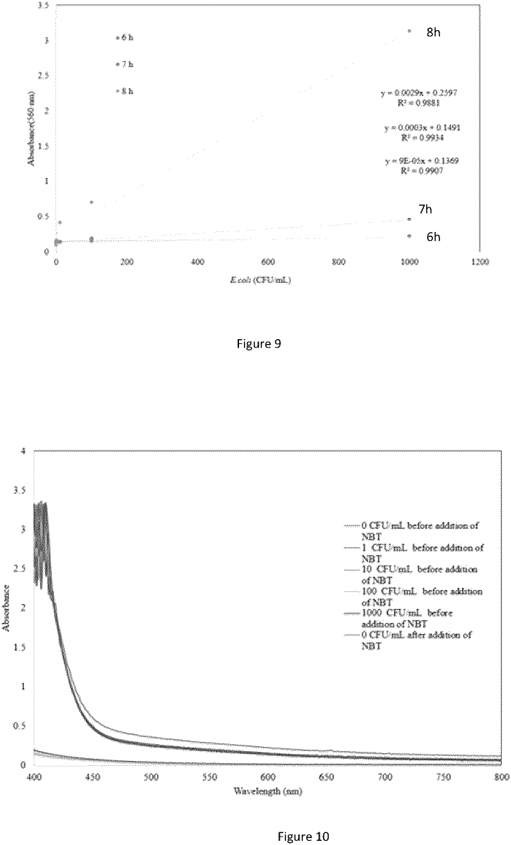

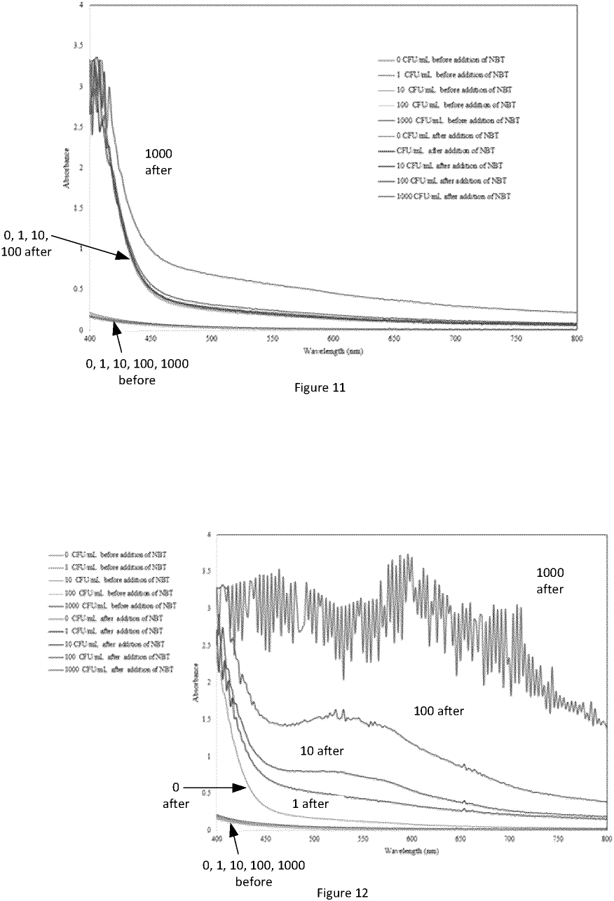

It was also determined that the enhanced color detection also improved the detection limit as low as 10 cfu/ml as detected with the use of a spectrophotometer without any filtration and concentration steps required such as shown in the charts of FIGS. 9 to 12. FIG. 9 shows a comparison of samples after the 6, 7 and 8 hours incubation period in the media incorporated with x-gluc at 37 C. At the end of respective incubation period NBT was added and absorbance at 560 nm was recorded after 10 minutes. FIG. 10 shows the incubation of different concentrations of E-coli in the media with x-gluc for 6 hours at 37° C. At the end of the incubation period, NBT was added and the spectrum was recorded after 10 minutes of the addition of the NBT. FIG. 11 shows the incubation of different concentrations of E-coli in the media with x-gluc for 7 hours at 37° C. At the end of the incubation period, NBT was added and the spectrum was recorded after 10 minutes of the addition of the NBT. FIG. 12 shows the incubation of different concentrations of E-coli in the media with x-gluc for 8 hours at 37° C. At the end of the incubation period, NBT was added and the spectrum was recorded after 10 minutes of the addition of the NBT.

In one embodiment of the disclosure, the disclosure may be seen as a method and system for probing or detecting β-glucuronidase activity as a marker for E.coli detection. The disclosure provides advantages over the traditional methods using x-gluc incorporated into a specific media. Addition of the second reagent allows colour development and detection within a predetermined period of incubation, well before it may be detected without the second reagent, or test formulation component. The disclosure provides a significant achievement for both detection limit and response time over current water testing methods.

In a further embodiment, after incubation, the water sample, or liquid sample may be filtrated and/or concentrated to further reduce the detection limit and/or detection response time. Furthermore, in another embodiment, the disclosure may include some level of filtration to enhance color change or possibly accelerate the color change but the disclosure is still directed at a two-stage process. This filtration may increase the initial bacterial number which may reduce the time required for the incubation period.

Applications of the disclosure may include beach/pool water testing; and/or wash water in food processing. The disclosure may also be integrated within an instrumental or tool.

In the preceding description, for purposes of explanation, numerous details are set forth in order to provide a thorough understanding of the embodiments. However, it will be apparent to one skilled in the art that these specific details may not be required. In other instances, well-known structures may be shown in block diagram form in order not to obscure the understanding.

The above-described embodiments are intended to be examples only. Alterations, modifications and variations can be effected to the particular embodiments by those of skill in the art without departing from the scope, which is defined solely by the claims appended hereto.

What is claimed is:

1. A method of detection of low level bacteria in a growth medium sample comprising:
adding a selective chromogenic substrate to the growth medium sample;
incubating the growth medium sample for a predetermined period of time; and
adding a dye reagent to the growth medium sample;
wherein if a color change in the growth medium sample is observed, a presence of bacteria is confirmed.

2. The method of claim 1 wherein the selective chromogenic substrate is 5-bromo-4-chloro-3-indoxyl-beta-D-glucuronide cyclohexylammonium salt (x-gluc); 1-5-Bromo- 4-Chloro-3-Indolyl Phosphate (BCIP) or 2-5-bromo-4-chloro-3-indolyl-β-D-galactopyranoside (x-gal).

3. The method of claim 1 wherein the dye reagent is nitroblue tetrazolium (NBT), tetrazolium-formazan substrates or resazurin substrates.

4. The method of claim 1 further comprising, before adding a selecting chromogenic substrate: placing an item of interest into the growth medium sample.

5. The method of claim 1 wherein adding a selective chromogenic substrate to the growth medium sample comprises:
adding the selective chromogenic substrate to a liquid sample.

6. The method of claim 5 wherein adding the selective chromogenic substrate to the liquid sample comprises:
adding the selective chromogenic substrate to a water sample.

7. The method of claim 1 further comprising, after adding a selective chromogenic substrate to the growth medium sample:
adding nutrients to the growth medium sample to enhance bacteria growth.

8. The method of claim 1 further comprising:
correlating an observed color change with a level of bacteria.

9. The method of claim 7 wherein correlating an observed color change comprises:
analyzing the liquid sample with a spectrophotometer or colorimeter.

10. The method of claim 1 wherein adding a selective chromogenic substrate to the growth medium sample comprises:
adding the selective chromogenic substrate to an agar gel sample.

11. A system for rapid detection of low level bacteria in a growth medium comprising:
a first test formulation component that is incubated for a predetermined period of time within the growth medium for enabling bacteria enzyme growth; and
a second test formulation component for adding to the incubated growth medium and first test formulation component.

12. The system of claim 11 wherein the first test formulation component comprises a selective chromogenic substrate.

13. The system of claim 12 wherein the selective chromogenic substrate comprises 5-bromo-4-chloro-3-indoxyl-beta-D-glucuronide cyclohexylammonium salt (x-gluc); 1-5-Bromo- 4-Chloro-3-Indolyl Phosphate (BCIP) or 2-5-bromo-4-chloro-3-indolyl-β-D-galactopyranoside (x-gal).

14. The system of claim 11 wherein the second test formulation component comprises a dye reagent.

15. The system of claim 14 wherein the dye reagent comprises nitroblue tetrazolium (NBT); tetrazolium-formazan substrates or resazurin substrates.

16. The system of claim 11 further comprising a set of nutrients for enhancing bacteria enzyme growth during the incubation period.

17. The system of claim 11 wherein the growth medium is in a liquid form.

18. The system of claim 11 wherein the growth medium is in a solid form.
